(19) United States
(12) Patent Application Publication
(10) Pub. No.: US 2014/0050674 A1
(43) Pub. Date: Feb. 20, 2014

(54) Method for Improving Penetration or Long Term Adhesion of Compositions to Dental Tissues and Compositions Usable in Said Method

(71) Applicant: Leo Tjaderhane, Oulun Yliopisto (FI)

(72) Inventor: Leo Tjaderhane, Oulun Yliopisto (FI)

(73) Assignee: University of Oulu, Oulun Yliopisto (FI)

(21) Appl. No.: 14/114,311

(22) PCT Filed: Apr. 30, 2012
(86) PCT No.: PCT/FI12/50422

§ 371 Date: Oct. 28, 2013

(30) Foreign Application Priority Data
Apr. 28, 2011 (FI) 20115404

(51) Int. Cl. A61K 6/027; A61Q 11/00

(52) U.S. Cl. CPC A61K 6/027; A61Q 11/00
USPC 424/49

(57) ABSTRACT

This invention concerns a method of improving penetration of compositions to dentin, enamel, dental pulp or cement and dental compositions for restoration or decoration of teeth, for use in pulp medication comprising, for root canal disinfection and/or obturation and for desensitizing. This invention also concerns uses of DMSO for improving the bond strength of dental composition and in preparing dental compositions. Further the invention concerns a new method of treating teeth so that penetration of components is increased.

### FIELD OF THE INVENTION

This invention relates to a new dental composition for restoration or decoration of teeth, for root canal treatment, for pulp medicament and for desensitizing. This invention relates also to a new method of improving long term adhesion of compositions or components to dental tissues and improving penetration of compositions to dentin, enamel, dental pulp or cement. This invention also relates to a use of dimethyl sulphoxide (DMSO) for improving the immediate and/or long term bond strength of dental composition, and uses of DMSO in preparing dental compositions having improved long term adhesion to dentin, enamel or cement. Further, this invention relates to a method of treating teeth comprising using DMSO to improve penetration of compositions.

The present invention is generally directed to compositions and methods for preparing the surfaces of teeth prior to their repair or restoration, including replacement of lost dental structures, cavity fillings, core build-ups, restorative cementations, and root canal treatments, and in particular to an improved immediate and long-term adhesion of the restorative or decorative materials to the dental tissues.

### DESCRIPTION OF RELATED ART

The restoration of dental structures, destroyed due to caries or wear, is commonly accomplished by the application of polymer-based adhesives to the dental structures. Improved adhesion leads to longer lasting restorations, reduced destruction of dental tissues, and reduced tooth sensitivity. To improve the immediate adhesion to dentin or enamel, tooth structures may be pretreated in various ways. Tooth may be treated with acid (“acid etching”) to remove the smear layer (a layer of debris forming on the tooth surfaces as a result of the cutting and grinding processes of e.g. cavity preparation), and to create a demineralized surfaces to dental structures. In enamel, etching increases the surface roughness and area, which improve the mechanical bond strength. In dentin surface, demineralization exposes dentin collagen matrix, which remains attached to the underlying mineralized dentin. When the acid is removed with water spray, the mineral part is replaced by rinse-water, resulting with 70 vol % water surrounding the 30 vol % collagen fibrils.

The etching step is followed by an application of a primer. Primers are generally surface-active compounds that exhibit both an affinity for dentin and adhesive resin systems and participate in the polymerization process, aiming to replace the water with adhesive monomer(s). After that, adhesive resin containing hydrophobic monomer(s) is applied followed by polymerization of monomers (three-step etch-and-rinse approach). Primer and adhesive may also be combined to exclude one step of the treatment (two-step etch-and-rinse approach). Alternatively, self-etching primers with acidic monomers to first treat the dentin surface instead of acid etching and then polymerize to adhesive are available. They can either include separate primer and adhesive (two-step self-etch approach) or primer-adhesive combined into one (one-step self-etch, or all-in-one approach).

During dental composite adhesion procedures, adequate removal of the smear layer and demineralization of enamel and dentin surface, good wetting, diffusion and penetration of adhesive monomers, and good polymerization of the resin components are all important. However, taking into account the aim to achieve long-lasting dental fillings, adhesive resin bonding to tooth structures is not as durable as it should be especially with dentin.

During the monomer infiltration phase of resin bonding, entire water component should ideally be completely replaced by resin monomers that polymerize to produce a hybridized biocomposite of resin reinforced with collagen fibrils, commonly known as the hybrid layer. The maximum depth of hybrid layers is only about 0.5 to 5 μm. The microscopic scale of these interfaces easily results with mistakes and failure of creating good bond in clinical work (Pashley et al. 2011). To ensure the proper penetration of adhesive monomer into the exposed dentin collagenous matrix, the matrix needs to be hydrated. If the matrix becomes dehydrated, interpeptide hydrogen bonds (H-bonds) cause the collagen fibers to stick together. The matrix collapses, and the interfibrillar spaces required as diffusion channels for resin monomer infiltration disappear (Pashley et al. 2011). The hybrid layer becomes poorly infiltrated with resin, and demineralized collagen fibrils will remain exposed. Resin retention—the bond strength—is very low (Pashley et al. 2011).

To keep the dentin matrix hydrated, adhesive systems contain solvents. In etch-and-rinse adhesive systems, the main functions of the solvents are to promote good monomer penetration into the collagen network of the demineralized dentin, and to re-expand the collapsed collagen network in case of overdrying. Water is the most common adhesive solvent. It is strongly polar with a high dielectric constant, capable of dissolving ionic lattices and polar compounds and forming strong H-bonds. In etch-and-rinse adhesives only water (due to its high dielectric constant) is capable to breaking the H-bonds between shrunken and collapsed demineralized dentin collagen fibers, resulting with re-expansion of the matrix and reappearance of interfibrillar spaces for monomer infiltration. In self-etch adhesives, water is essential for ionization of acidic monomers, which would do the demineralization (Van Landuyt et al. 2007). However, water is a poor solvent for organic compounds, including monomers which are usually hydrophobic. Therefore, water is often used together with a secondary solvent (co-solvent), usually ethanol or acetone. The role for the co-solvents is to “push out” water, allowing the monomers to penetrate the spaces between collagen fibers.

It is difficult to find the ideal combination of solvents in primers or primer/adhesives. Water is needed to keep the collagen network open, but excess water in the adhesive resin compromises the bond strength of adhesives (‘overwet phenomenon’) (Tay et al. 1998). The high boiling temperature, low vapour pressure and fluid movement out of dentinal tubules into the hypertonic comonomer mixtures make water difficult to remove from adhesive solutions after application on the tooth (Van Landuyt et al. 2007). Increasing the co-solvents in the etch-and-rinse adhesives may jeopardize the proper expansion of the collagen network and therefore may prohibit the penetration of adhesive monomers between the collagen fibers, resulting with failure in bonding. In self-etch adhesive systems, high concentration of co-solvents results with fewer protons formed, resulting with improper demineralization.

The wetness of dentin makes it necessary to use hydrophilic monomers, such as hydroxyethyl methacrylate (HEMA), in the primer or primer-adhesive to penetrate the water-rich dentin collagen matrix. Hydrophilic monomers and solvent(s) together significantly improve the adhesives' wetting behaviour.

Unfortunately, in uncured state hydrophilic monomers readily absorb water, which may lead to dilution of the monomers to the extent that polymerization is inhibited. Also in cured state, hydrophilic monomers still take up water (Van Landuyt et al. 2007) leading to subsequent hydrolytic degradation of polymer (Carrilho et al. 2005). Depending on several factors, such as hydrophilic behaviour, also other methacrylate monomers are susceptible to hydrolysis in aqueous solutions (Van Landuyt et al. 2007).

Good evaporation of the solvents with air-drying after application onto tooth tissue should facilitate the removal of remaining solvent from the adhesive. Complete evaporation is, however, difficult to achieve within clinically reasonable drying time. With all the traditional solvents, remaining solvent may jeopardize polymerization due to dilution of the monomers, and may increase permeability of the adhesive layer. The conversion rate (degree of polymerization of the monomers) is an important determinant of the physico-mechanical strength of the resulting polymer. Polymerization is inhibited by several factors, including the presence of intrinsic water from dentin and the presence of residual solvents in the adhesive. Regions within the polymerized hybrid layer that are water-rich and resin-poor (the areas of incomplete resin infiltration) are called nanoleakage, and they increase in size with aging, reducing the bond strength and durability (Pashley et al. 2011). Apart from low mechanical strength, low conversion rate also results in higher permeability, increased water sorption, increased nanoleakage, and degradation of the tooth-composite bond (Van Landuyt et al. 2007). Collagen that is left exposed undergoes hydrolytic degradation caused by the collagenolytic enzymes present in dentin, namely matrix metalloproteinases (MMPs) and cysteine cathepsins (Tersariol et al. 2010, Pashley et al. 2011). The inhibition of these enzymes is considered an important step in prevention of loss of dental adhesive bond strength (Breschi et al. 2008, Pashley et al. 2011).

In intact condition, pulp tissue is encased within hard tissue formed by enamel, cementum and dentin. However, the external irritation due to caries, wear or toxic substances, induces inflammation in pulp tissue. This initially local inflammation may progress into widely-spread inflammation and necrosis, if the cause of the irritation is not removed, leading finally to pulp necrosis and loss of tooth vitality. The treatment of pulp tissue, to down-regulate inflammatory reaction, reduce pain or protect the tissue from progressive necrotic damage, should therefore be initiated at the early phases of the inflammation (Cooper et al. 2010), e.g. under relatively small caries lesion or during the normal restorative procedures. Since the inflamed pulp in these cases is still protected by dentin, especially at the early stages of the inflammation, local medication is difficult. With a suitable carrier to penetrate the remaining dentin, various medicaments and substances, aiming to heal the pulp and/or induce the reparative processes protecting the dental pulp against external damage. Such medicaments or substance could include, but are not limited to, growth factors, anti-inflammatory agents, enzyme inhibitors, antimicrobial agents, localized pain medicaments, and so on.

When microbes penetrate the dentin and reach the dental pulp, this eventually leads to infection of the entire root canal system and inflammatory response in the bone around the tip of the root (apex: apical periodontitis). The elimination of microbes from the root canals requires endodontic treatment, combining mechanical debridement of infected soft tissue and chemical disinfection with antimicrobial irrigation solutions (irrigants) and medicaments. The most commonly used irrigants are sodium hypochlorite (NaOCl), chlorhexidine and iodine, and the most common interappointment medicament is calcium hydroxide (Ca(OH)₂) (Zehnder 2006). In long-standing root canal infections, bacteria can invade the adjacent dentin via open dentinal tubules. The penetration of the root canal irrigants and medicaments into the tubules is essential to reach and eliminate microbes from the tubules, improving the healing of periapical inflammation and reducing the risk of reinfection of the root canal system (Zehnder 2006). The infiltration of dentin by chemical components from aqueous solutions occurs via diffusion rather than direct liquid exchange (Zehnder 2006). Improving the diffusion, possibly together with reducing the surface tension, by combining currently used intracanal irrigants and medicaments (including at least, but not limited to, NaOCl, chlorhexidine, iodine, Ca(OH)₂) would improve the effectiveness of irrigants and medicaments, as they would better reach into dentinal tubules and accessory canals.

There is thus a need for further improving stability fastening of dental compositions and components. There is also a need for improved means of delivering compositions or components, such as medicaments, into tooth structures without need of extensive mechanical invasion. This invention meets these needs.

### OBJECTS AND SUMMARY OF THE INVENTION

It is an aim of the invention to provide compositions for restoration or decoration of teeth.

Further it is aim to provide a method for improving penetration of compositions to dentin, enamel, dental pulp or cement and to improve the bond strength of dentinal adhesives and to provide a method to improve long-term adhesion of compositions or components to dental tissues.

Still further aim is to provide compositions and methods for use in dental care.

Particularly, the aim is to provide means and compositions improving the stability of dental fillings.

The above-described drawbacks and disadvantages are alleviated by a use of dimethyl sulphoxide (DMSO; aka methanesulfinylmethane, methylsulfinyl-methane, methyl sulfoxide) as an active ingredient and possibly to at least partially replace the conventional solvent to be used in adhesive procedures. DMSO should be present in quantities effective to provide improved penetration of adhesive monomer(s) into the porosities and surface irregularities of enamel and dentin, including the spaces within exposed dentin collagen matrix; and in quantities effective to provide efficient, long-term inhibition of the function of enzymes present at or reaching into the hybrid layer. These DMSO quantities are generally in the range from about 0.001 to about 30%. This composition can be provided as a separate component, or included into an etchant or primer/adhesive of the adhesive system, for ease of application and storage. The composition may accordingly include other solvents, monomer(s), polymerization initiator(s), desensitizing agent(s), antimicrobial agent(s), enzyme-inhibiting agent(s), fluoride or fluoride source(s), or filler component(s). Shortly, these and other objects are achieved by the present invention as hereinafter described and claimed.

The first aspect of the invention is a dental composition for restoration or decoration of teeth. According to the invention the composition comprises
(a) at least one monomer; and optionally
(b) a solvent and
(c) one or more of the group consisting of initiators, inhibitors, desensitizing agents, fillers, silane coupling factors, cross-linking agents, dyes and acids,
   wherein the composition further comprises 0.0001-1.9% DMSO.

In this connection the term “dental composition” means any composition suitable for use in therapeutic or cosmetic treatment of teeth. In addition to traditional compositions used in restoration of teeth, especially preparing the surfaces of teeth prior to their repair or restoration, the dental compositions also include compositions for treating pulp or root canal, desensitizing agents, toothpaste and adhesives for use in attaching e.g. jewel for decorative purpose. It must be noted that the dental compositions for restorative and adhesive uses (including root canal sealers and obturation materials) disclosed here are not meant to be biodegradable and they do not include fat or essential amounts of cyanoacrylates.

In this connection term “a solvent” or “a further solvent” includes single solvents and also mixtures of various solvents (solvent systems) that are suitable for use in dental care.

The second aspect of the invention is a method of improving long term adhesion of compositions or components to dental tissues. According to the invention the method comprises the step(s) of including DMSO into one or more composition used.

The third aspect of the invention is a dental composition for use in pulp medication comprising one or more constituents selected from the group consisting of MMP inhibitors, fluoride, anti-microbial agents, anti-inflammatory agents, growth factors, pain medicaments and optionally a solvent (or solvents). According to the invention the composition further comprises 0.0001-80% DMSO.

The fourth aspect of the invention is a dental composition for root canal disinfection and/or obturation comprising
(a) at least one disinfectant or demineralizing agent; and optionally
(b) additional antimicrobial or demineralizing agents;
(c) detergents.

According to the invention, the composition further comprises 0.0001-1.9% DMSO.

The fifth aspect of the invention is a dental composition for root canal obturation comprising an irrigant or primer and a sealer; or a sealer. According to invention, the composition further comprises 0.0001-1.9% DMSO.

The sixth aspect of the invention is a dental composition for desensitizing comprising two or more constituent(s) of the group consisting of ions or salts, protein precipitant(s); dentin sealer(s), and optionally one or more of the group consisting of further solvent or solvents, detergents and homeopathic medications. According to invention the composition further comprises 0.0001-80% DMSO.

The seventh aspect of the invention is the use of DMSO for improving the long term bond strength of dental composition.

Further aspects of the invention are the use of DMSO in preparing dental compositions having improved long term adhesion to enamel, dentin or cement, in preparing dental compositions for pulpal treatment or root canal treatment and in preparing dental compositions for desensitizing.

Another further aspect is a method of improving penetration of compositions to dentin, enamel, dental pulp or cement. According to the invention the method comprises including DMSO into said composition.

Still further aspect of the invention is a method of treating teeth. According to invention the method comprises using DMSO to improve long term adhesion of compositions or components therein and optionally to inhibit MMP's.

The preferred embodiments of the invention are disclosed in the dependent claims.

### BRIEF DESCRIPTION OF THE DRAWINGS

FIG. 1. Concentration-dependent decrease in contact angle of water on dentin surfaces treated with different DMSO concentrations.
A) The contact angles during the measurement time of angle changes (up to 60 s after dropping the liquid). Each value represents mean of 10 measurements.
B) Contact angles (mean±standard deviation) 1 s and 60 s after dropping the liquid.

FIG. 2. Acid-etched dentin is treated with 5% DMSO, and contact angle measurements from 1 to 60 s are performed with 1 s interval. DMSO treatment significantly improves both immediate (1 s) and final (60 s) wettability of dentin, leading to better penetration of adhesive monomers into exposed dentin collagen matrix and dentinal tubules. This results with improved immediate bond strength, and better long-term protection of exposed collagen matrix against enzymatic degradation. The angles represent the mean of 10 separate measurements.

FIG. 3. SEM images of adhesive on dentin surface without (control: A) and with 30% DMSO (B) treatment. In control sample, the surface is almost completely covered with the adhesive (A), while most of the DMSO-treated sample had open dentinal tubules exposed (B), demonstrating the penetration deep into tubular dentin due to improved wettability. In control, very small areas of dentin surface left exposed (areas within dotted lines in C: higher magnification of the area marked with white square in A), with few dentinal tubule orifices detectable (C, white arrowheads). In DMSO-treated sample, the situation is practically reverse: minor areas are covered with the adhesive, while most of the surfaces open dentinal tubules can be clearly seen (D: higher magnification of the area marked with white square in B).

FIG. 4. Pulpal fluid flow into the hybrid layer during adhesive procedures and composite build-up. The samples were subjected to water with silver nitrate (AgNO₃) particles using physiological pulpal pressure (20 cm H₂O pressure) from the pulp chamber (at the bottom of the image) The uppermost light area is composite filling.
A) Control sample with dentin surface treated with water before adhesive application. Silver deposits in the hybrid layer (a) are present in the hybrid layer (b). Silver deposits can also be seen in the dentin-pulp border (c) as well as in the dentinal tubules (d) through which they have entered into the hybrid layer.
B) Sample with 30% DMSO-treated surface. Strong silver staining can be seen in the dentin-pulp chamber border and immediate dentin (c); while dentinal tubules and hybrid layer (b) contain no silver particles.

FIG. 5. DMSO effect on dentinal fluid flow during the making of adhesive layer and building the composite layer. Ctr: control group (water). N=5 in each group. *: significantly different from all DMSO groups (p<0.05, ANOVA with Tukey's test).

FIG. 6. SEM images of dentin-composite filling interface of control sample (A) and tooth treated with 30% DMSO before adhesive application (B). The dentin-adhesive interface was exposed by treating the surface with acid and sodium hypochlorite to remove smear layer and perform surface demineralization and deproteinization.

FIG. 7. The effect of dentin DMSO treatment on immediate bond strength with four commercially available adhesives belonging to three different adhesive systems. Scotchbond SE: Adper™ Scotchbond™ SE Self-Etch Adhesive; Silorane: Filtek™ Silorane System Adhesive Self-Etch Primer and Bond; SB1: Adper™ Single Bond 1XT Adhesive; SBMP: Adper™ Scotchbond™ Multi-Purpose Plus Adhesive (all materials from 3M ESPE). **: statistically significant difference, p<0.01: independent-samples t-test.

FIG. 8. The effect of dentin DMSO treatment on long-term durability of bond strength with three adhesive systems. four commercially available adhesives belonging to three different adhesive systems. Scotchbond SE: Adper™ Scotchbond™ SE Self-Etch Adhesive; Silorane: Filtek™ Silorane System Adhesive Self-Etch Primer and Bond; SB1: Adper™ Single Bond 1XT Adhesive; SBMP: Adper™ Scotchbond™ Multi-Purpose Plus Adhesive (all materials from 3M ESPE). Asterisks indicate statistically significant differences: *: p<0.05; **: p<0.01; ***: p<0.001: independent-samples t-test.

FIG. 9. Percentual changes in bond strengths with DMSO treatment of dentin. All the values are related to the respective control sample, given the value of 100% (the dotted line as a reference).

FIG. 10. The effect of 0.003% DMSO treatment on immediate and long-term nanoleagake, as detected with AgNO₃ impregnation method.
A, B) Examples of FESEM images of control (A) and DMSO-treated (B) samples in which the nanoleakage has been detected with AgNO3. The top part of the images is composite filling material; the dark band below is the adhesive layer; and the bottom part is tubular dentin. Silver accumulation, indicating nanoleakage, can be seen as white particles in the interface between dentin and the adhesive, where the hybrid layer is located.
C, D) The respective samples after 6 month storage. Markedly larger surface area demonstrates silver particle accumulation in the control samples compared to the immediate control sample (A) or 6-month DMSO sample (B).
E) Percentage of the hybrid layer with nanoleakage, as detected with silver particle accumulation into the hybrid layer. (*: statistically significant difference to both immediate control and 6-month DMSO: p<0.05, Mann-Whitney U-test).

FIG. 11. DMSO effect on fluid flow through root canals with endodontic obturation, using three different commercially available sealers. Ctr: control group (sterile saline); DMSO: irrigation of root canals with 5% DMSO immediately before obturation. N=10 in each group. *: significantly different from the control, p<0.05; *: significantly different from the control, p<0.01 (Mann-Whitney U-test).

FIG. 12. Matrix metalloproteinase (MMP) inhibition by DMSO.
A) DMSO effect on gelatinase MMP-2 and -9 activities, as observed with gelatin zymography. Dark bands in the gel indicate MMP activity; the darker the band, the stronger the activity. APMA: 4-aminophenylmercuric acetate, a common activator for MMPs.
B) The relative densitometric values of DMSO inhibition of gelatinases in zymographic analysis presented in A. The data represents mean±standard deviation (SD) of three individual experiments.

FIG. 13. DMSO effect on gelatinase MMP-2 activity, as measured with commercial enzyme activity kit EnzChek. The bars represent fluorescent units released from fluorescently labelled gelatin due to gelatinolytic enzyme activity during 24 h incubation period. Different letters above the bars indicate the groups with statistically significant differences (p<0.05: ANOVA with Tukey's method). PA: 1,10-phenatroline, MMP inhibitor included into the EnzChek kit.

FIG. 14. The effect of DMSO on hydroxyproline (HYP) release from demineralized dentin beams. “1 week” indicates HYP release during the 1st week of incubation, and “Total” is the total amount of HYP released during the entire 4 week of the experiment. *: significantly different from the control group, p<0.05; **: significantly different from the control group, p<0.01 (Kruskall-Wallis and Mann-Whitney U-test).

### DESCRIPTION OF PREFERRED EMBODIMENTS

According to the invention DMSO is used in various dental compositions for improving the penetration of compositions to the structures of the tooth and reducing water content in the teeth and thereby preventing destructive microbial and enzymatic activity. In restorative or decorative uses DMSO can be included in an etchant, separate wetting solution, primer, adhesive or any combination thereof, such as self-etching primer, primer-adhesive composition, self-etching primer-adhesive or one step self-etching cement. DMSO can also be used to improve properties of desensitizing agents, a solution for treating dental pulp, a solution for cleaning or disinfecting the root canal, or as a separate solution or as a component of a root canal sealer to improve the penetration of the sealer or the obturation material. It has surprisingly been found that DMSO has capability to enhance penetration of dental composition to tooth. Further advantage of using DMSO is its MMP-inhibiting property.

DMSO can be included in the dental composition or it can be added to said composition before using it in the treatment. In one embodiment DMSO is added to a commercial composition before use. This is possible as DMSO is compatible with most of the conventional compositions/components used in dental compositions and small amount needed for improving the penetration does not compromise the properties of the composition.

In restorative or adhesive purposes enhanced penetration increases the immediate bond strength. DMSO also increases the long term bond strength. Further, as DMSO acts as MMP inhibitor and reduces the amount of water at dentin-bonding interface also the long term bond strength is greatly increased. Thus, the DMSO is added into restorative or decorative composition (in which bond strength is important) to a final concentration of 0.0001 to 50%, preferably 0.0005 to 30%, more preferably 0.001 to 10%, still more preferably 0.001 to 4.5, still more preferably 0.001 to 1.9%, still more preferably 0.001 to 1% and most preferably 0.001 to 0.5%, e.g. about 0.003%. Such DMSO content is suitable for improving the penetration of etching, priming and adhering components to a tooth. Examples of restorative or decorative purposes are replacement of lost dental structures, cavity fillings, core build-ups, restorative cementations, including methods for preparing the surfaces of teeth prior to their repair or restoration.

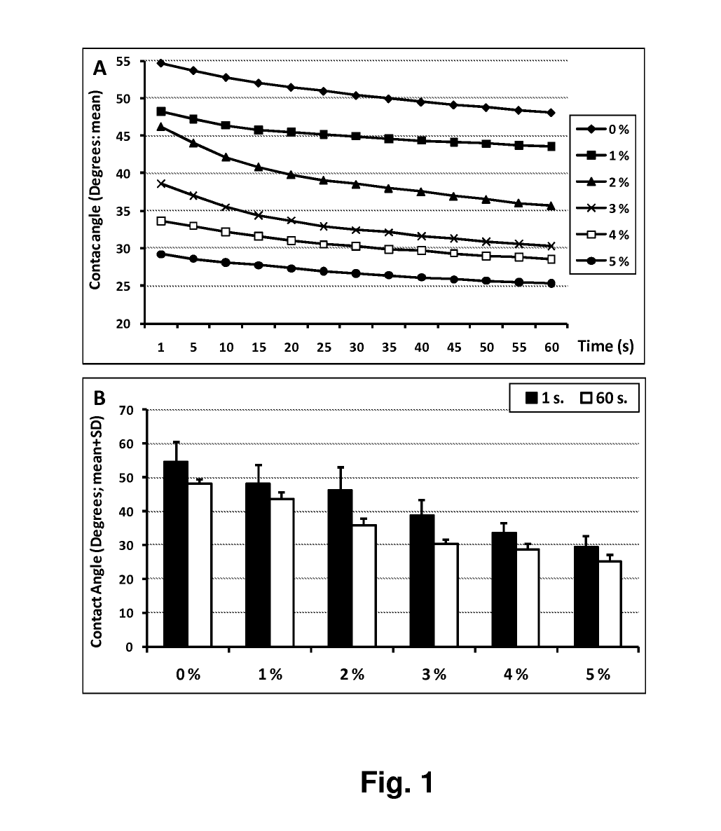

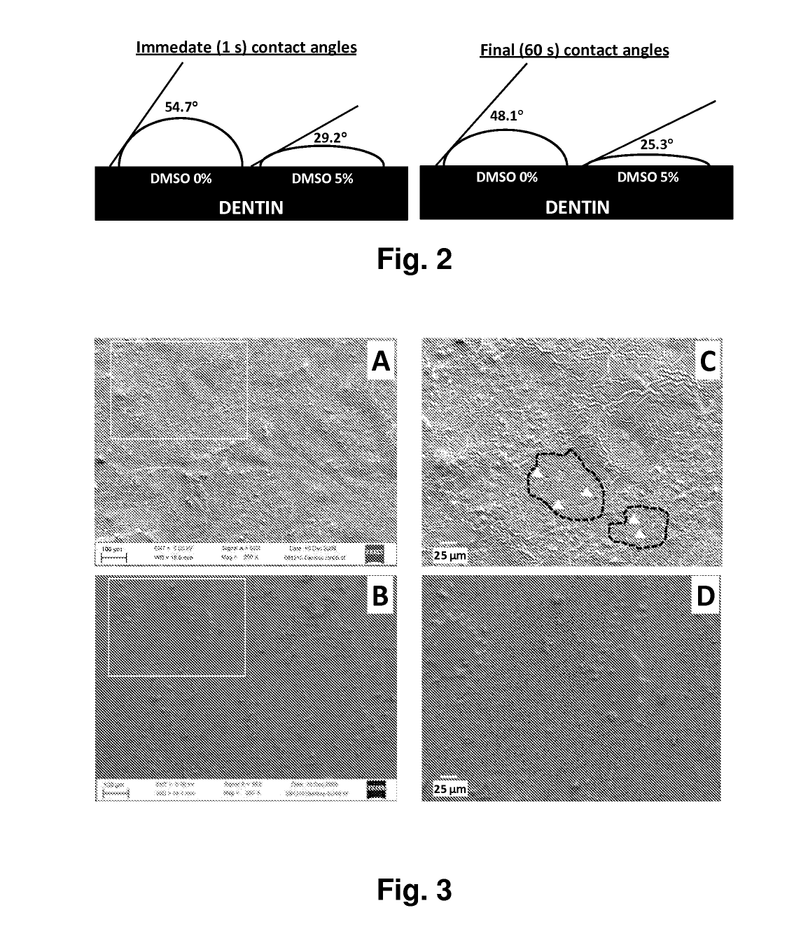

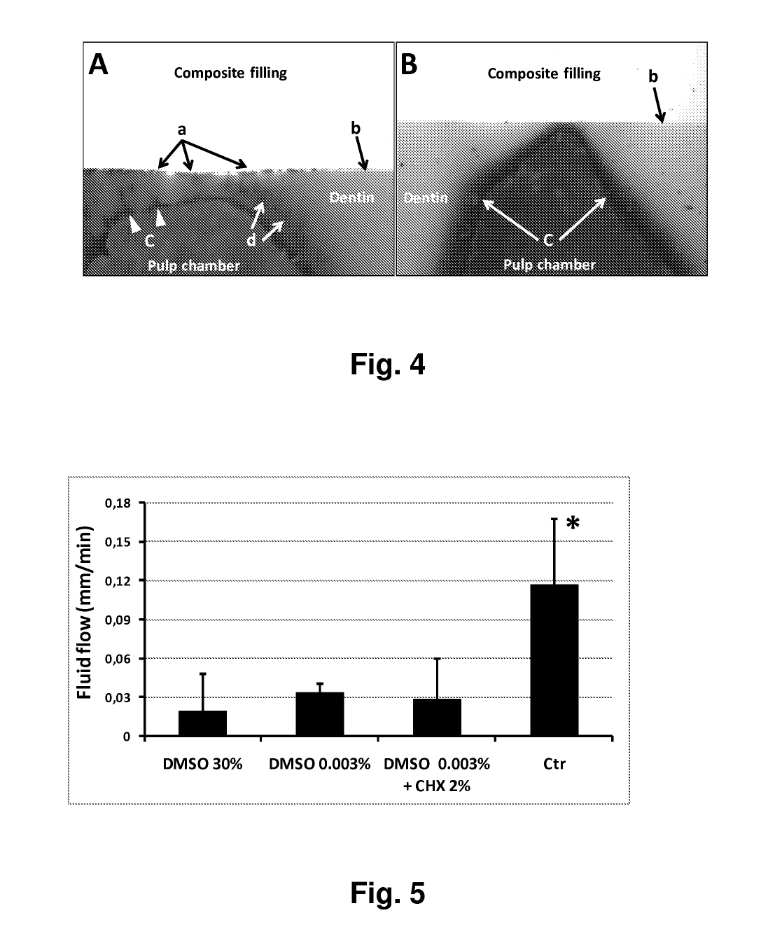

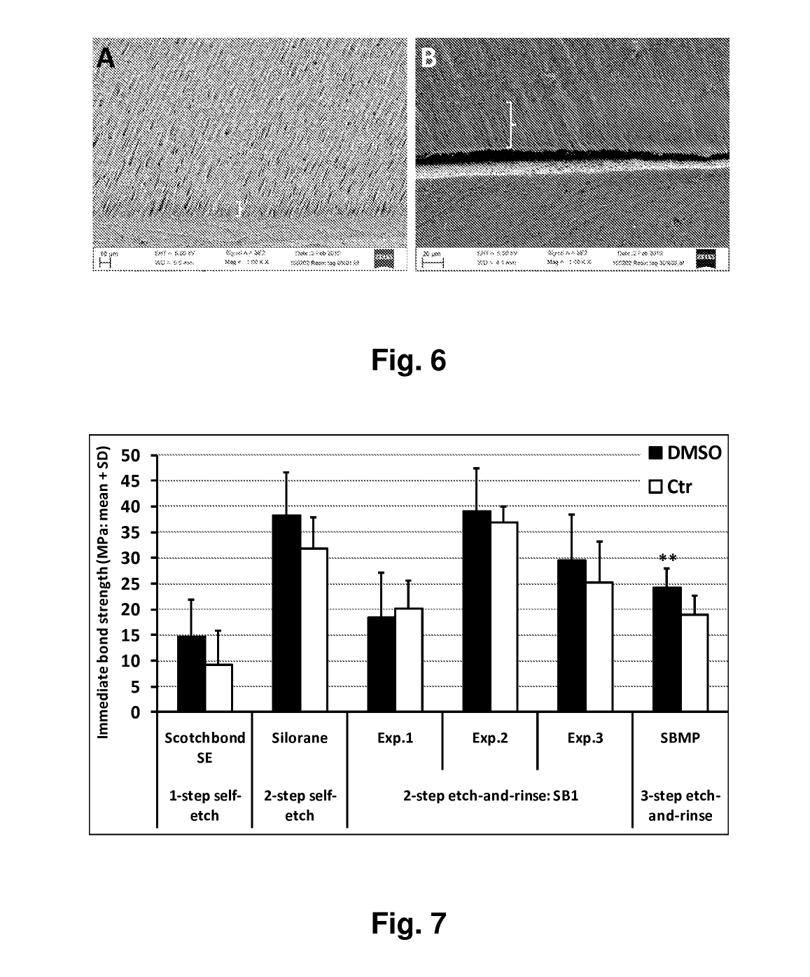

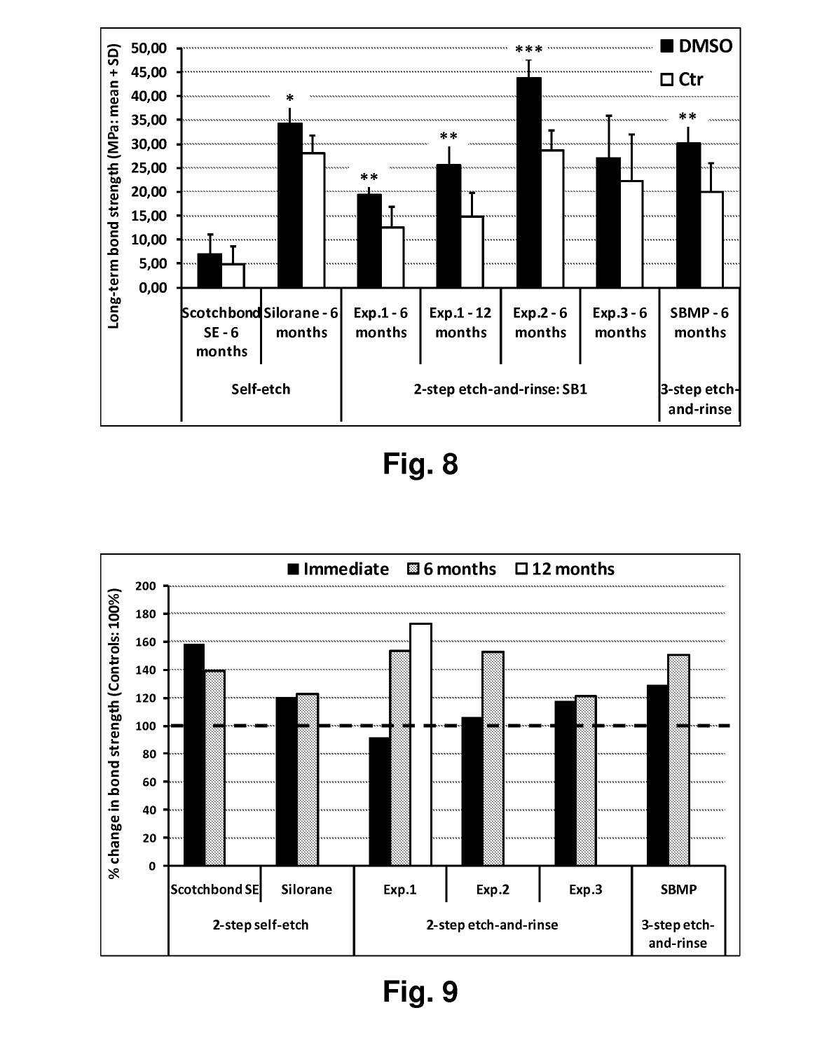

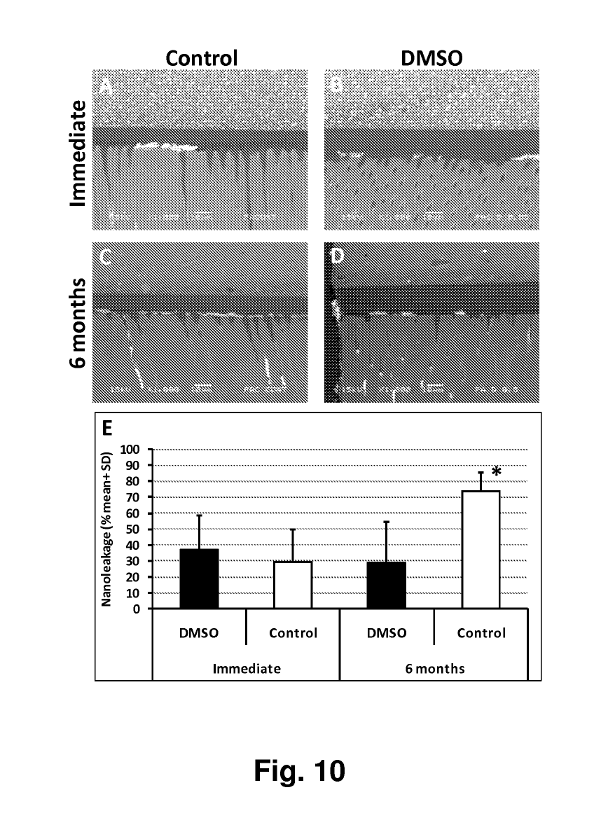

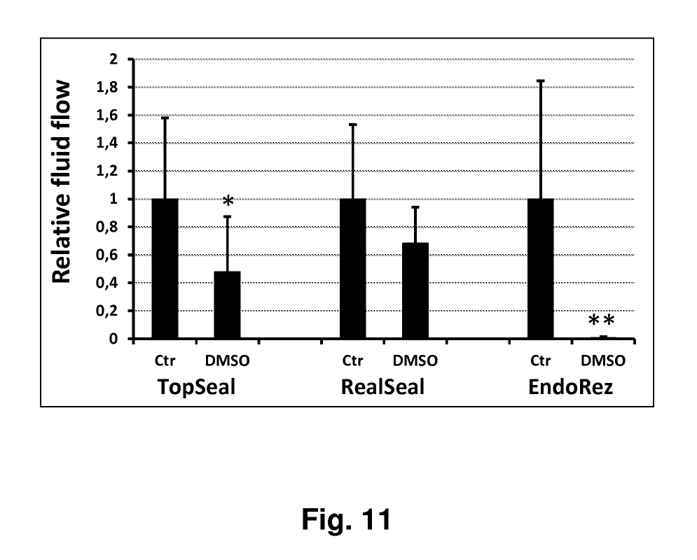

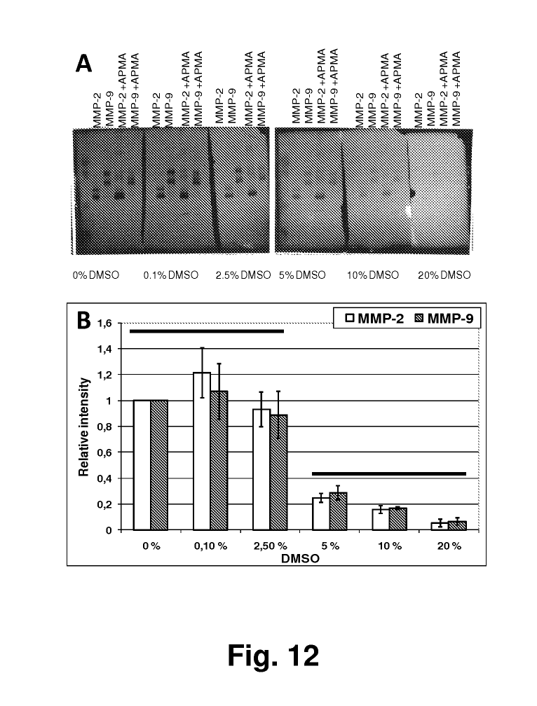

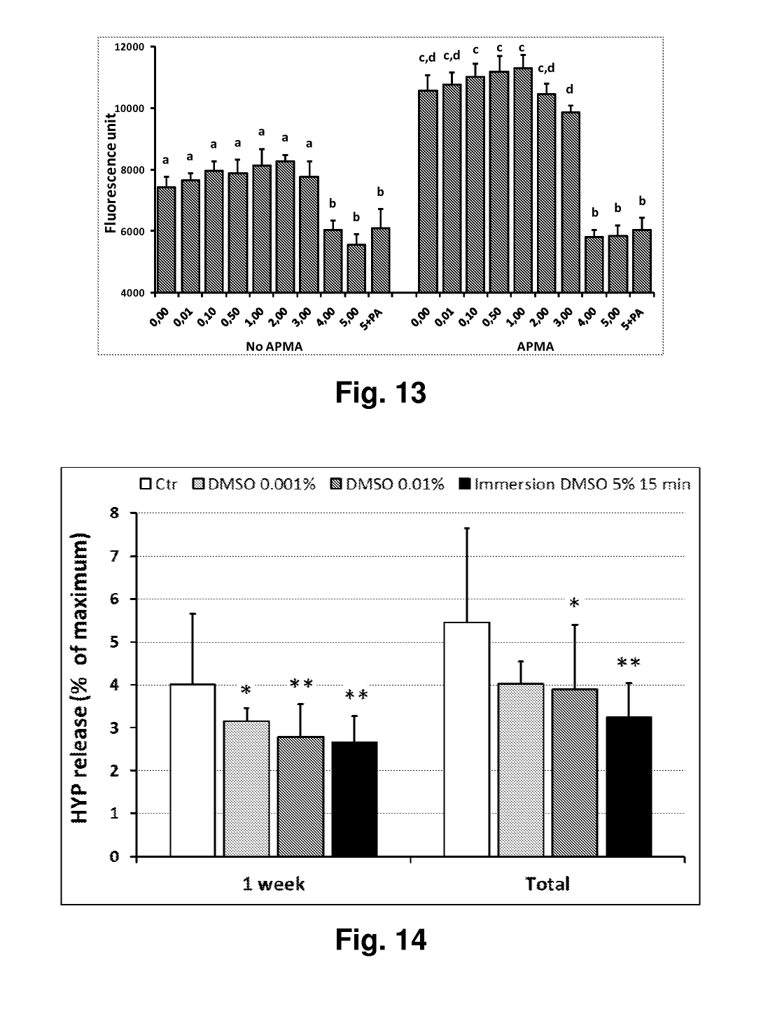

The first advantage that can be gained with the use of DMSO in connection with adhesive systems is the higher immediate bond strength. When the effect of DMSO was examined, it was noted that treatment of acid-etched dentin surface with DMSO dose-dependently improved the wettability of the surface (FIGS. 1, 2; Table 1). Increased wettability improves the penetration of dental adhesive into dentinal tubules and between the exposed collagen fibrils in dentin surface (FIG. 3). When the effect of DMSO on fluid flow in dentinal tubules was examined, it was noticed that the fluid flow from the pulpal side into the adhesive layer and restorative material was greatly reduced by DMSO (FIG. 4 and FIG. 5). This causes improved penetration of primer/adhesive monomers into the tissue (FIG. 6), thus improving the immediate bond strength by up to 27.9% (Table 2, FIG. 7).

The second advantage is the better long-term stability of the bond strength (FIG. 8, FIG. 9). This is probably caused by the better initial penetration of primer/adhesive that would better encapsulate the dentin collagen fibers within polymerized adhesive layer, thus protecting them from hydrolytic degradation (FIG. 10); and by the inhibition of dentinal collagen matrix metalloproteinases (MMPs). When the effect of DMSO on MMPs was examined, the MMPs were found to be inhibited up to 94.7% with 20% DMSO, and up to 75.3% with 5% DMSO (FIG. 12, FIG. 13). When the effect of DMSO on demineralized dentin degradation by enzymes present in the collagenous matrix, significant decrease in collagen degradation was observed (FIG. 14).

In this connection the component to be attached can be a direct dental restoration, an indirect dental restoration (an inlay or onlay), a crown, a sealant, a laminate, an orthodontic bracket, a jewel, or respective.

One particular embodiment is a method of therapeutic or cosmetic restoring of a tooth, wherein the penetration of the adhesive to the dentin is improved and thereby the bonding of filling material or filling is enhanced. Thus, in one embodiment a filling material is introduced. Suitable amount of DMSO to be used in the method is 0.0001 to 1.9%, preferably 0.0005 to 1.9%, more preferably 0.001 to 1.9%, still more preferably 0.001 to 1.5, still more preferably 0.001 to 1% and most preferably 0.001 to 0.5%, e.g. about 0.003%.

In one embodiment of the invention the method of improving the penetration comprises the steps of
(a) etching; i.e. the treatment of dental hard tissue to remove the surface smear layer and mineral component, to increase the enamel bonding surface and to expose dentinal collagen fibrils to allow bonding
(b) priming; i.e. the treatment of wet enamel or dentin surface to accept adhesive monomers
(c) applying adhesive; i.e. the layer of adhesive resin to connect the primer-treated hybrid layer and the component to be bonded
(d) introducing a component to be bonded to tooth surface (e.g. dental composite, filling material, or luting cement),
   which steps may be either separate or be combined and the method is characterized by adding DMSO in a dental composition in at least one of the steps. In more detail,
   steps a. to d. can be separate steps, or a. and b., b. and c. or a., b. and c. can be combined. In case of self-etching cements, a single composition can be used and all the method steps can be combined.

In one embodiment the steps a. to d. are carried out to restore a damaged tooth. In another embodiment said steps are carried out for the cosmetic restoration or decoration of the tooth.

In one embodiment of the invention DMSO is included in one-step filling composition such as self-etching cement.

A dental composition for restoration or decoration of teeth can comprise, in addition to at least one monomer and DMSO, constituents selected from other solvents, polymers and copolymers, initiators, inhibitors, desensitizing agents, fillers, silane coupling factors, cross-linking agents, dyes, and acids as discussed below. Preferably such a composition does not contain H₂NO₃.

Dental composition can be wetting agent, demineralizing agent (etchant), primer, adhesive or a filling material or a combination thereof such as self-etching primer, primer-adhesive composition, or self-etching primer-adhesive. Primer and adhesive compositions are especially suitable.

Examples of suitable demineralizing agents are phosphoric acid, ethylenediaminetetraacetic acid (EDTA), citric acid and maleic acid.

The monomer can be, without restricting to those, selected from group consisting of 4-acryloyloxyethyl trimellitate anhydride (4-AETA), 4-acryloylethyl trimellitic acid (4-AET), 2-acrylamido-2-methyl-1-propanesulfonic acid (AMPS), bis[2-(methacryloyl-oxy)ethyl]phosphate (Bis-MEP), ethoxylated bisphenol A glycol dimethacrylate (bis-EMA), bisphenol A diglycidyl methacrylate (bis-GMA), biphenyl dimethacrylate or 4,40-dimethacryloyloxyethyloxycarbonylbiphenyl-3,30-dicarboxylic acid (BP DM), di-2-hydroxyethyl methacryl hydrogenphosphate (di-HEMA phosphate), dimethylaminoethyl methacrylate (DMAEMA), ethyl 2-[4-(di-hydroxyphosphoryl)-2-oxabutyl]acrylate (EAEPA), ethyleneglycol dimeth-acrylate (EGDMA), glycerol dimethacrylate (CDMA), glycerol phosphate dimethacrylate (GPDM), 1,6-hexanediol dimethacrylate (HDDMA), 2-hydroxy-ethyl methacrylate (HEMA), 2-hydroxyethyl methacryl dihydrogenphosphate (HEMA-phosphate), hexafluoroglutaric an hydride-glycerodimethacrylate adduct (HFGA-GMA), 2-hydroxypropyl methacrylate (HPMA), methacrylic acid (MA), 2,4,6 trimethylphenyl 2-[4-(dihydroxyphosphoryl)-2-oxa-butyl]acrylate (MAEPA), 11-methacryloyloxy-1,10-undecanedicarboxylic acid (MAC-10), 10-methacryloyloxydecyl dihydrogenphosphate (10-MDP), methacryloyloxydodecylpyridinium bromide (MDPB), 4-methacryloyloxyethyl trimellitate anhydride (4-META), 4-methacryloyloxyethyl trimellitic acid (4-MET), methyl methacrylate (MMA), mono-2-methacryloyloxyethyl phthalate (MMEP) which is sometimes also called phtalic acid monomethacrylate (PAMA), N-methacryloyl-5-aminosalicylic acid (5-NMSA or MASA), N-phenylglycine glycidyl methacrylate (NPG-GMA) and N-tolylglycine glycidyl methacrylate or N-(2-hydroxy-3-((2-methyl-1-oxo-2-propenyl)oxy)propyl)-N-tolyl glycine (NTG-GMA).

In addition to DMSO, the etchant/primer/adhesive composition may contain other solvents. For detailed applications, a suitable solvent completely wets and diffuses into the conditioned surfaces in clinically acceptable time (on the order of about 15 to 180 seconds). Examples of polar solvents suitable for use in conjunction with the present invention include, but are not limited to, methanol, ethanol, propanol and other low molecular weight alcohols such as tert-butanol; low molecular weight ketones such as acetone and methyl ethyl ketone; polar aprotic solvents such as dimethylformamide, di-methylacetamide, 1-methyl-2-pyrrolidinone; or combinations and mixtures thereof. Water or a mixed solvent system of water and ethanol or water and acetone are preferred. Adhesive in three-step systems (using three separate dental compositions) may also be without the solvents, such as e.g. Adper™ Scotchbond™ Multi-Purpose plus dental adhesive (3M ESPE, Seefeld, Germany). An adhesive is a special case of dental composition where typically no solvent is used.

Examples of suitable polymers are ormocer (organically modified ceramics) polymers, polyethylene glycol dimethacrylate (PEGDMA), pentamethacryloyl-oxyethylcyclohexa-phosphazene monofluoride (PEM-F), dipentaerythritol pentaacrylate monophosphate (PENTA), 2-(methacryloyloxyethyl)phenyl hydrogenphosphate (phenyl-P), pyromellitic diethylmethacrylate or 2,5-dimethacryloyloxyethyloxycarbonyl-1,4-benzenedicarboxylic acid (PMDM), pyromellitic glycerol dimethacrylate or 2,5-bis(1,3-dimethacryloyloxyprop-2-yloxycarbonyl)benzene-1,4-dicarboxylic acid (PMGDM), polyalcenoic acid copolymer, tetramethacryloyloxyethyl pyrophosphate (Pyro-EMA), butan-1,2,3,4-tetracarboxylic acid di-2-hydroxyethylmethacrylate ester (TCB), triethylene glycol dimethacrylate (TEGDMA), trimethylolpropane trimethacrylate (TMPTMA) and urethane dimethacrylate or 1,6-di(methacryloyloxyethylcarbamoyl)-3,30,5-trimethyl-hexaan (UDMA).

Examples of suitable initiators, inhibitors and desensitizing agents are butylhydroxytoluene or butylated hydroxytoluene or ,2,6-di-(tert-butyl)-4-methylphenol (BHT, inhibitor), benzoylperoxide (BPO, redox initiator), benzenesulfinic acid sodium salt (BS acid, redox initiator), camphorquinone or camphoroquinone or 1.7.7-trimethylbicyclo-[2,2,1]-hepta-2,3-dione (CQ, photo-initiator), tri-n-butyl borate (TBB, initiator), N,N-di-(2-hydroxy-ethyl)-4-toluidine (DHEPT, co-initiator), bis(2,4,6-trimethylbenzoyl)-phenylphosphine oxide (Irgacure 819), 4-methoxyphenol or monoethyl ether hydroquinone (MEHQ, inhibitor), 2-(ethylhexyl)-4-(dimethylamino)benzoate (ODMAB, co-initiator), 1-phenyl-1,2 propanedione (PPD, initiator/co-initiator), Lucirin TPO (TPO, photo-initiator, BASF), 2-hydroxy-4-methoxybenzophenone (UV-9, photo-initiator), and glutaraldehyde (desensitizing agent; cross-linker; disinfectant), and proanthocyanidin (cross-linker; collagenase inhibitor).

Examples of suitable fillers, silane coupling factors, dyes, acids and cross-linking agents are g-methacryloxypropyltrimethoxysilane (Coupling factor A174), full reaction type pre-reacted glass-ionomer fillers (F-PRG), maleic acid, sodium fluoride (NaF), disodium hexafluorosilicate (Na₂SiF₆), polyhedral oligomer silsesquioxanes (POSS nano-particulates), other nanoparticles and nanoclusters, silica acid and Thymol blue.

In one embodiment of the invention the final amount of DMSO in the dental composition is 0.0001 to 1.9, preferably 0.0005 to 1% and most preferably 0.001 to 0.5%, e.g. about 0.003%.

In one embodiment the dental composition is for pulpal medicament and comprises 0.0001-80% DMSO; preferably 0.0005-30%, more preferably 0.001 to 10% and most preferably 0.003 to 4.5% and optionally solvent, typically aqueous solvent such as water, physiological saline, and one or more antimicrobial agent(s), enzyme inhibitors (especially MMP-inhibitor), fluoride, stabilizers, anti-inflammatory agents, pain medicaments and growth factors. Improved penetration allows e.g. reaching microbes in the tubules and reduces need of mechanical dentin removal for bacterial elimination, or penetration of anti-inflammatory agents into inflamed pulp tissue. Further, DMSO inhibits destructive MMP-activity in tubules. When used as a carrier, it is important that DMSO does not possess cytotoxic effects on dental pulp.

At the first phase of root canal treatment, tooth pulp chamber and root canals are cleaned mechanically with root canal instruments, using irrigants such as sodium hypochlorite (NaOCl) (so-called chemomechanical preparation). In addition to removing pulp tissue remnants, NaOCl acts as a detergent, dissolves organic tissue and kills microbes, all beneficial effects in root canal treatment. In addition to or instead of NaOCl, other agents such as ethylenediaminetetraacetic acid (EDTA), hydrogen peroxide (H₂O₂), chlorhexidine, ethanol or other alcohols, or physiological saline can be used. The product may further contain detergents or other lubricating agents, such as glycerol or carbowax (polyethylene glycol).

In one embodiment of the invention the dental composition is the irrigation solution for chemomechanical preparation, and comprises NaOCl or other irrigation agents and DMSO. Improved penetration of irrigant into the areas of root canal systems that cannot be reached with instruments, and dentinal tubules, improves the tissue dissolving and antimicrobial effects of the irrigant.

At the end of the root canal mechanical preparation, the final irrigation is performed with EDTA and often also with chlorhexidine (Zehnder 2006). The purpose of EDTA is to remove the smear layer and to open up the dentinal tubules to allow the penetration of chlorhexidine into dentinal tubules to improve the antimicrobial effect. Instead of EDTA, also citric acid or other acidic or chelating agents can be used.

In one embodiment of the invention the dental composition is a solution for removing the smear layer and opening the dentin tubules. The composition comprises EDTA or other acidic or chelating agent and DMSO; and optionally other constituents, such as antibiotics. Improved access of chlorhexidine and/or other antimicrobial agent(s) into dentinal tubules will improve the antimicrobial effects of the said constituents.

After final irrigation, the root canal system can either be filled with temporary antimicrobial compositions (medicaments), or permanently obturated. The most commonly used root canal medicament (composition for root canal treatment) is calcium hydroxide (Ca(OH)₂), but also other agents with long-lasting antimicrobial effect, such as povidone-iodine (PVPI) or iodine potassium-iodide (IKI, Lugol's iodone) can be used. The aim is to achieve long-lasting (days, weeks, months) antimicrobial effect in cases of e.g. complicated root canal infections.

In one embodiment of the invention the dental composition is an antimicrobial composition (medicament) comprising DMSO and Ca(OH)₂, PVPI, IKI or respective long-term antimicrobial agent. The improved penetration of antimicrobial agent into the areas of root canal systems that cannot be reached with instruments, and dentinal tubules, improves the tissue dissolving and antimicrobial effects.

During the final obturation, the sealer is first applied into the root canal system, followed by the main obturation material, usually gutta percha or respective semi-rigid cones. The role of the sealer is to fill the areas unreachable by main material, to seal the gap between the main obturation material and root canal walls, to improve the seal by penetrating into the open dentinal tubules and in some cases to adhere mechanically and/or chemically to dentin and obturation material (FIG. 11).

In one embodiment of the invention the dental composition is a solution comprising DMSO and water, physiological saline or other basic solution; and optionally other antimicrobial, anti-inflammatory or other constituents (such as chlorhexidine). The solution, when used prior to application of the sealer, will improve the penetration of the sealer into unaccessable areas and dentinal tubules. Alternatively, the composition may be a sealer including DMSO, with the same benefit as above.

Dentin sensitivity to thermal changes (especially cold), sweet and acidic foods or beverages, is caused by the fluid movements in exposed and open dentin tubules. Dentin sensitivity is a common dental problem. It is treated with so-called desensizing agents that cover, obturate or plug the dentinal tubules, thus preventing the fluid movement and dentin sensitivity. The desensitizing agents can be applied either by the dental professionals, or they can be included into commercial products available for consumers (such as oral rinses or toothpastes). In one embodiment the composition is for dentin desensitizing and comprises 0.0001 to 80%, preferably 0.001 to 30% and more preferably 0.003 to 4.5% and even 0.003 to 1% DMSO and conventional compositions. Lower concentrations may be suitable for consumer products. Ions/salts, protein precipitants, phytocomplexes, dentin sealers and optionally homeopathic medications are used for covering or plugging dentinal tubules. DMSO improves the desensitation effect by improving the penetration of desentizing compositions/components into the tubules.

Examples of ions/salts suitable for use in covering or plugging dentinal tubules are aluminium salts, ammonium hexafluorosilicate, bioactive glass (e.g. SiO₂, NaO₂), calcium hydroxide, calcium carbonate, calcium phosphate, calcium silicate, dibasic sodium citrate, potassium oxalate, silicate, fluorosilicate, sodium monofluorophosphate, sodium fluoride, stannous fluoride, sodium fluoride/stannous fluoride combinations, strontium acetate with fluoride and strontium chloride.

Examples of protein precipitants suitable for dentinal treatment are formaldehyde, glutaraldehyde, silver nitrate (AgNO₃), strontium chloride hexahydrate, zinc chloride and proanthocyanidins.

Optional phytocomplexes suitable for dentinal treatment include Rhubarb rhaponicum and Spinacia oleracia, and homeopathic medications may include Plantago major, Propolis and grape seed extracts (polyphenols, proanthocyanidins), amongst others.

Suitable dentin sealers are for example glass ionomer cements, dental adhesives and composites, resinous dentinal desensitizers, varnishes, sealants, methyl methacrylate and other monomers.

A special example from everyday life is toothpaste with desensitizing properties (Sensodyne, [GlaxoSmithKline] product family is an example of a commercial preparation). Addition of DMSO further improves the desensitizing properties.

In addition to the above defined components also potassium nitrate can be used for nerve desensitization.

One embodiment of the invention is the use of DMSO for improving the bond strength of dental composition. DMSO can be used as a separate step, for example in wetting, or it can be included to conventional preparations.

A further embodiment of the invention is the use of DMSO in preparing dental compositions having improved ability to penetrate to dentin, enamel or cement. In a further embodiment DMSO is used in preparation of a primer or adhesive composition or a combination thereof. DMSO improves both the long term and short term bond strength.

In one embodiment of the invention the DMSO is used in dental pulp treatment at concentration of 0.0001-80% DMSO; preferably 0.0005-30%, more preferably 0.001 to 10% and most preferably 0.003 to 4.5%. Relatively high DMSO content may be beneficial in pulpal treatment allowing penetration of medicament without extensive mechanical debridement.

Further, DMSO has an MMP inhibiting effect thereby still improving the results of treatment. Other possible components of such composition are MMP inhibitors, anti-inflammatory agents, growth factors, fluoride and pain medicaments.

In a still further embodiment DMSO is used in the preparation of the composition for root canal treatment. As discussed above, DMSO can be used in all steps of root canal treatment and remarkable advantages are achieved.

In a still further embodiment DMSO is used in the preparation of desensitizing compositions including toothpaste and desensitizers commonly used by professional practitioners. It is further evident that DMSO can be used both in disinfection and obturation processes, wherein DMSO enhances penetration of occluding bodies deeper into the dentin tubules thereby reducing dentin sensitivity.

In the above defined embodiments DMSO can be added as one constituent to a commercial composition, or it can be added to a composition by the dentist or dental assistant before using it.

One embodiment of the invention is a method of restoring teeth wherein the method comprises the step(s) of
(a) etching
(b) priming
(c) applying adhesive
(d) introducing a component,
   wherein steps a. to c. can be separate steps. a. and b., b. and c. or a., b. and c. can be combined and wherein DMSO is used as a component in at least one of the steps a. to d. DMSO can be used as a separate step or included to a composition used. The advantages of using DMSO have been discussed above and are mainly based to its ability to improve penetration of compositions (components) and to inhibit MMPs.

The invention is illustrated by the following non-limiting examples. It should be understood, however, that the embodiments given in the description above and in the examples are for illustrative purposes only, and that various changes and modifications are possible within the scope of the invention.

### EXAMPLES

### Example 1

### Contact Angle Measurements

### Materials and Methods

To minimize variation between samples all measurements were conducted using the following series of procedures: (1) the dentin surface was sanded using 500 grit abrasive sandpaper to produce consistent smooth surface; (2) etched with phosphoric acid etchant (Scotchbond Etchant, 3M ESPE) for 15 to achieve a uniform, smear layer-free dentin surface; (3) rinsed with deionized water for 15 s; (4) the dentin surface was exposed to deionized water with 0% (control), 1%, 2%, 3%, 4% or 5% (vol) DMSO in deionized water for 60 s; (5) the dentin surface was dried with filtered air for 60 s; (6) contact angle measurements were conducted using deionized water.

Six intact human third molars were used after extraction from students at the university health center as a part of their dental treatment. The teeth were stored in 2% sodium azide at +4° C. until preparation. The occlusal enamel surface was cut off with a precision saw, exposing the superficial dentin. Dentin surfaces were polished with 500 grit sandpaper to achieve even smear layer-covered surface similar to clinical situation. The dentin surface was then etched with phosphoric acid etchant for 15 s with gentle scrubbing, and rinsed with deionized water for 15 s. Then they were fitted to their moulds and exposed to one of six possible concentrations of deionized water/DMSO for 60 seconds. After exposure the dentin surface was dried with compressed air for 60 s.

For sessile drop contact angle measurements an optical surface tension/contact angle meter (KSV Ltd. CAM 200, KSV Instruments Ltd., Helsinki, Finland) was used, with a CCD-Camera. A Hamilton plunger-type syringe, with standard Kel-F Hub-51 mm needle, was used for the droplet application on dentin surface. The average drop volume was 6.2 (±2.5) μl. Each sample was measured ten times. In between measurements, the dentin surface was briefly rinsed with deionized water and lightly air dried before being exposed to a water/DMSO solution and dried. Images were captured at 1 Hz frequency for 61 seconds using an automated trigger, data was collected and contact angles were calculated using a computer and CAM Series Software (KSV Instruments Ltd.).

### Results

Contact angle decreased with increasing vol-% DMSO in a linear fashion, demonstrating an increase in dentin surface wettability with increasing DMSO concentration (Table 1; FIG. 1). The improved wettability creates noticeably better spreading of liquid onto surface (FIG. 2), suggesting improved penetration of adhesive into the acid-etched dentin surface with exposed collagen network. The effect is also very fast: the mean difference in angle between 5% DMSO and control (0% DMSO) in the first measurement (1 second after drop) is already 25.5°. The relative contact angle remained the same throughout the measurement: during the whole recording period (60 s, the contact angle being recorded every second) the mean contact angle with 5% DMSO was 52.92% (SD 0.27%; maximum 53.38% and minimum 52.52%) of that measured in control samples.

TABLE 1
The effect of DMSO concentration in the pretreatment solution on
water contact angle on demineralized dentin surface. The percentage after
dash indicates the contact angle in relation to the control at the same time
point (100%).

| | Contact angle (degrees; mean and SD) | | |
|---|---|---|---|
| DMSO | 1 s after drop | 10 s after drop | 60 s after drop |
| 0% | 54.7 (5.9)^{A}/100% | 52.8 (3.7)^{A}/100% | 48.1 (1.3)^{A}/100% |
| 1% | 48.3 (5.4)^{A,B}/88.2% | 46.4 (3.6)^{B}/84.8% | 43.6 (2.1)^{B}/79.6% |
| 2% | 46.2 (6.8)^{B}/84.5% | 42.1 (4.0)^{B}/77.1% | 35.7 (2.1)^{C}/65.3% |
| 3% | 38.6 (4.7)^{C}/70.5% | 35.5 (3.2)^{C}/64.8% | 30.2 (1.4)^{D}/55.3% |
| 4% | 33.7 (2.7)^{C,D}/61.5% | 32.2 (2.6)^{C,D}/58.9% | 28.5 (1.7)^{D}/52.2% |
| 5% | 29.2 (3.4)^{D}/53.4% | 28.2 (3.1)^{D}/51.5% | 25.3 (1.9)^{E}/46.2% |

The groups with different upper case letter are significantly different from each other (ANOVA with Tukey's post-hoc test; p < 0.05).

### Example 2

### Adhesive Penetration

### Materials and Methods: SEM Analysis

After acid etching, the control sample was left slightly moist, as described in the instructions of the adhesive used; experimental sample was slightly dried with compressive air, rewetted with 30% DMSO and left slightly moist as with control sample. Then the adhesive (SB1, 3M ESPE) was similarly applied to both surfaces, solvent was evaporated with compressed air for 15 s, and the adhesive was light cured. Half of the samples were left unfilled to examine the adhesive layer on the dentin surface. Half were restored with a 2-mm layer of composite (Filtek Supreme XT, 3M ESPE) and light-cured, then cut perpendicular to the filled surface to expose the hybrid layer. The cut surfaces were then treated with phosphoric acid for 15 s and NaOCl for 5 min to remove the smear layer and expose the dentin-adhesive interface. All the samples were then dehydrated in the increasing series of alcohol, sputtered with gold, and subjected to examination with SEM.

### Materials and Methods: Silver Nitrate Impregnation

The two samples were glued to a plexiglass plate with cyanoacrylate glue. Pressure of 20 cm H₂O was provided to the pulp chamber during bonding via a 16 gauge metal tube running through the plexiglass. Samples were bonded using the same protocol as for the fluid flow measurements, one with the new solvent protocol and the other according to the manufacturer's instructions. 20 cm H₂O pressure was used 24 hours later to deliver aqueous AgNO₃ into the pulp chamber for 24 hours. Samples were then cut along the longitudinal axis of the tooth into 1 mm thick slabs and viewed with a light microscope.

### Materials and Methods: Fluid Flow Measurements

Forty intact, caries-free human third molars were used in this study. Mid-coronal dentin discs were acquired by using a low speed precision saw with water coolant and polished on both sides with 160 grit abrasive paper with constant water spray to reach the final thickness of 0.65 mm±0.1 mm. Both sides of the disc were etched with 35% phosphoric acid for 15 s to achieve a uniform, smear layer-free dentin surface. The apical side was then glued to a wide end of a pipette tip with cyanoacrylate adhesive. The pipette head was then cut 1 cm from the tip, filled with deionized water, and pushed inside silicone tubing with an inner diameter of 2.0 mm using silicone grease as sealant to prevent leakage. The same silicone grease was used in every joint of the fluid-filled system.

The flow of deionized water through dentin samples was measured using a Flodec device (DeMarco Engineering, Geneva, Switzerland). In Flodec, an optical system constantly follows the position of an air bubble in 10 μL microtube, and the movement is recorded by a specific computer program. The device is able to detect nanoliter-scale changes of fluid volume, an important feature in measuring slight variations in specimen flux (De La Macorra et al. 2002). Prior to adhesive application, a constant 0.4 mm/min fluid flow was determined for each sample separately to eliminate the effect of sample variances on fluid flow rate.

Five samples were selected at random for each group, and were treated before bonding with one of the DMSO- or control solutions. The bonding procedure was performed with the above-mentioned 0.4 mm/min flow rate for each sample. The bonding agent was Scotchbond 1XT (a new version of SB1, also known as Single Bond Plus, 3M ESPE).

The procedure was as follows. Each sample was exposed to one of the DMSO- or control solutions for 15 s, gently dried using cotton pellets, and adhesive was applied twice during a 15 second period with constant agitation with the brush. Pressurized air was then used to evaporate solvents until the adhesive surface stopped moving, and light cured for 10 seconds (Elipar S10 LED Curing Light, 3M ESPE). Composite filling material (Filtek Supreme XT Universal Restorative, 3M ESPE) was applied onto the treated surface in two 2-mm increments and separately light cured for 20 s. A prefabricated mold was used to ensure the matching size and location of composite build-up on the sample.

### Results

SEM images presented in FIG. 3 show adhesive on dentin surface without (control: A) and with 30% DMSO (B) treatment. In control sample, the surface is almost completely covered with the adhesive (A). Very small areas of dentin surface are left exposed (areas within dotted lines in C: higher magnification of the area marked with white square in A), with few dentinal tubule orifices detectable (C, white arrowheads). In DMSO-treated sample, the situation is practically reverse: most of the surface has open dentinal tubules exposed (B). Minor areas are covered with the adhesive, while most of the surface's open dentinal tubules can be clearly seen (D: higher magnification of the area marked with white square in B).

FIG. 4 shows pulpal fluid flow into the hybrid layer during adhesive procedures and composite build-up, as demonstrated with the penetration of AgNO₃ particles. In control sample (FIG. 4A) subjected to fluid flow (from the bottom of the image) with silver nitrate (AgNO₃) particles, and dentin surface treated with water before adhesive application. Accumulation of silver deposits (a) in the hybrid layer (b) indicate marked fluid flow from the pulp chamber into the hybrid layer. Silver deposits can also be seen in the dentin-pulp border (c) as well as in the dentinal tubules (d) through which they have entered into the hybrid layer. In the sample treated with 30% DMSO prior to adhesive application, strong silver staining can be seen in the dentin-pulp chamber border and immediate dentin (c); while dentinal tubules and hybrid layer (b) contain no silver particles.

FIG. 5 shows DMSO effect on dentinal fluid flow during the making of adhesive layer and building the composite layer. The data clearly demonstrates that DMSO is capable of inhibiting the dentinal fluid flow into the hybrid layer after acid etching and during adhesive application and composite build-up. The data also demonstrates the ability of DMSO (even in low concentrations) to introduce chlorhexidine into the dentin, as the samples treated with 0.003% DMSO-2% chlorhexidine does not differ from other DMSO-containing solutions.

The SEM image in FIG. 6A indicates that approximately 10 μm hybrid layer—a normal hybrid layer thickness—is formed in control sample (white bracket in A). With 30% DMSO (FIG. 6B), approximately 40 μm layer is rendered acid-resistant, indicating intensive penetration of adhesive into demineralized dentin organic matrix and even beyond.

In summary, the data clearly demonstrates that DMSO treatment is capable for significant reduction of pulpal fluid flow into the hybrid and adhesive layers. This indicates the potential for both improved initial bond strength (better penetration resulting with increase in bonding surface) and better long-term bond durability due to extensively good protection of collagen from enzymatic degradation. The experiments also demonstrate that lower DMSO concentrations are equally sufficient to exclude excess water from the hybrid and adhesive layers than higher (30%) concentration. The finding is important, since the effectiveness with the very low concentration significantly widens the range of potentially usable concentrations in clinical work. Since combining low-concentration DMSO with 2% chlorhexidine (the agent currently most often used to improve the durability of dentin bond strength via MMP inhibition [Pashley et al. 2011]) did not affect DMSO's ability to prevent fluid flow, DMSO may be used in conjunction with chlorhexidine or other agents aiming at the improvement of dentin bond longevity. These agents may include, but not be limited to, specific and non-specific MMP inhibitors, chlorhexidine, benzalkonium chloride and other quaternary ammonium compounds and salts; glutaraldehyde, carbodiimide, proanthocyanidin and other collagen cross-linkers; cross-linking monomers; and other agents aiming to better immediate and/or long-term bond strength.

### Example 3

### Microtensile Bond Strength Testing

Since all the data indicated better replacement of water and improved penetration of adhesive into the hybrid layer and underlying dentin, microtensile bond strength evaluations were performed to examine whether the effect would also be seen in the bond strength.

### Materials and Methods

The teeth (five to eight teeth per group, depending on the experiment) were sectioned under water cooling coronally to remove occlusal enamel and to expose flat dentin surface, and at the dentin-enamel junction. Exposed dentin surface was ground with 180-grit abrasive paper to create uniform smear layer. In the control samples, the adhesives were used as recommended by the manufacturer. Briefly, with self-etch adhesives in control group, the surface was scrubbed with water using extensively moist cotton pellet, gently dried to leave the dentin surface slightly moist, and then the dentin surfaces were treated first with primer and then with adhesive; in the experimental group 0.003% DMSO was scrubbed to the surface for instead of water, with all other steps identical to those performed with the controls. With etch-and-rinse adhesives, in the control group the dentin surface was first acid-etched with 37% phosphoric acid, rinsed, gently dried with compressed air to achieve slightly dry surface, then water was scrubbed into the surface with cotton pellet for 30 s and gently dried, leaving the surface slightly moist, and then adhesive was used as per manufacturer's recommendations; in experimental group, instead of water the surface was scrubbed with 0.003% DMSO after acid-etching and drying, with all other steps similar to those with controls. 4-mm composite resin restoration (Filtek Supreme XT) was then built on top of the adhesive-treated dentin with 1-mm increments that were separately polymerized. All adhesives and the composite resin were unaltered commercially available products from 3M ESPE (Seefeld, Germany).

In one experiment, DMSO was added directly into adhesive to examine the effect of mixing DMSO with the adhesive on immediate bond strength. Twenty drops (470 μg) of Scotchbond Universal 2-step etch-and-rinse adhesive (3M ESPE) were placed in a dispensing well (provided as part of the adhesive kit) and 4.7 μg of pure DMSO was added with Hamilton syringe and thoroughly mixed with an adhesive application tip for 30 s to ensure complete dispersion of the DMSO into the adhesive. The resultant adhesive contained about 1% DMSO (w/w). The dispensing well with the adhesive was covered with a small plastic single-use dental dispensing cup to prevent evaporation and premature polymerization. The teeth were acid-etched, rinsed, and gently dried as described above and according to the adhesive manufacturer's instructions. The adhesive was applied with gentle rubbing for 20 s and gently dried to evaporate excess solvents and polymerized with light as instructed by the manufacturer. The composite resin build-up was then performed as described above. The control teeth were treated similarly except that the adhesive did not contain DMSO.

The teeth were stored in artificial saliva (Carrilho et al. 2007a) for 24 h to allow postoperative polymerization of the adhesive and composite to take place. Then the teeth were longitudinally sectioned across the bonded interface in sections perpendicular to the pulpal wall with a diamond saw, to produce a series of 0.9 mm×0.9 mm×8 mm beams (Carrilho et al. 2007a). Bond strength was tested within a week from making the filling. Approximately half of the beams were tested immediately, while the rest were stored for six or 12 months to evaluate the durability of bond strength. In bond strength testing, each specimen was individually fixed to a testing jig with cyanoacrylate glue and subjected to tensile load at a crosshead speed of 0.5 mm/min until failure. The force required to fracture the sample and the exact dimensions of the fracture site were recorded, and tensile bond strength (in MPa) was calculated.

### Results

Of six separate experiments with separate dentin treatment with DMSO, all but one (Experiment #1 with 2-step etch-and-rinse adhesive SB1, Single Bond 1) demonstrated higher immediate bond strengths (Table 2; FIG. 7). The difference was statistically significant with 3-step etch-and-rinse adhesive (SBMP: Scotchbond Multipurpose Plus. **: p<0.01).

TABLE 2
Immediate bond strengths of four different adhesive systems in six
separate experiments, representing three different bonding approaches
(self- etch adhesives and two kinds of etch-and-rinse adhesives).
“Increase in bond strength” indicates the percentual change
in the mean bond strength caused by the use of DMSO.

| | | | Etch-and rinse adhesives | | | |
|---|---|---|---|---|---|---|
| | Self-etch adhesives | | 2-step | | | |
| Group/ | Scotch- | | SB1: | SB1: | SB1: | 3-step |
| adhesive | bond SE | Silorane | Exp. 1 | Exp. 2 | Exp. 3 | SBMP |
| DMSO 0.003% | 14.2 (6.2) | 38.2 (6.1) | 18.4 (5.5) | 39.1 (3.1) | 29.5 (8.9) | 24.3** (3.8) |
| Control | 10.7 (6.7) | 32.0 (8.4) | 20.1 (8.7) | 36.9 (8.2) | 25.2 (7.9) | 19.0 (3.7) |
| Increase | 32.7% | 19.4% | −8.5% | 6.0% | 17.1% | 27.9% |
| in bond | | | | | | |
| strength | | | | | | |

All adhesives were commercially available adhesives from 3M ESPE (Seefeld, Germany). The values represent the bond strengths (mean and standard deviation).
SB1: SingleBond 1;
SBMP: Scotchbond MultiPurposePlus.
“Exp. 1-3” represent three separate experiments with SingeBond 1.
**bond strength significantly higher than in controls, p < 0.01, t-test.

When DMSO was used as mixed directly into adhesive, higher immediate bond strength was also observed. With DMSO, the immediate bond strength was 29.8±5.7 MPa (mean and standard deviation, respectively), and in controls 26.1±5.4 MPa, respectively. The resulting increase in immediate bond strength was 10.8%.

After storage for six or 12 months, DMSO-treated samples demonstrated higher bond strength in all experiments with separate dentin treatment with DMSO described above. In all but two (1-step self-etch adhesive Scotchbond SE, and Experiment #3 with 2-step etch-and-rinse adhesive Single Bond 1) DMSO treatment produced statistically significantly higher long term bond strengths (Table 3; FIG. 8).

TABLE 3
The effect of dentin DMSO treatment on long-term bond strength
with three adhesive systems. “Increase in bond strength” indicates the
percentual change in the mean bond strength caused by the use of DMSO.

| | Self-etch | | | | | | |
|---|---|---|---|---|---|---|---|
| | adhesives | | Etch-and rinse adhesives | | | | |
| | Scotch- | | 2-step | | | | 3-step |
| Group/ | bond SE | Silorane | SB1: Exp. 1 | SB1: Exp. 1 | SB1: Exp. 2 | SB1: Exp. 3 | SBMP |
| adhesive | 12 months | 12 months | 6 months | 12 months | 6 months | 12 months | 12 months |
| DMSO | 6.9 (4.2) | 34.3* (4.1) | 19.3** (4.4) | 25.6** (5.7) | 43.8*** (3.8) | 26.9 (9.0) | 30.1** (5.1) |
| 0.003% | | | | | | | |
| Control | 5.0 (3.7) | 28.0 (3.7) | 12.6 (4.3) | 14.8 (4.9) | 28.7 (4.0) | 22.1 (9.9) | 19.9 (6.0) |
| Increase | 38.0% | 22.5% | 53.2% | 73.0% | 52.6% | 21.7% | 51.3% |
| in bond | | | | | | | |
| strength | | | | | | | |

All adhesives were unaltered commercially available products from 3M ESPE (Seefeld, Germany). The values represent the bond strengths (mean and standard deviation).
*p < 0.05;
**p < 0.01;
***p < 0.001: independent-samples t-test. The abbreviations as in Table 2.

In summary, the findings clearly demonstrate that DMSO improves the immediate and long-term bond strength of adhesive procedures. Percentual changes in bond strengths with DMSO treatment of dentin are shown as FIG. 9. The figure clearly demonstrates that especially the long-term (six and 12 months) bond strengths are improved when the respective controls are used as a reference (100%).

### Example 4

### Scanning Electron Microscopy for Nanoleakage Evaluation

### Materials and Methods

Three sticks, prepared for the microtensile testing, from DMSO and one from control group immediately and after 6-month storage were used for nanoleakage evaluation, using method described by Klein-Júnior et al. (2008). The sticks were placed in the ammoniacal AgNO₃ in darkness for 24 h, rinsed thoroughly in distilled water, and immersed in photo developing solution for 8 h under a fluorescent light to reduce silver ions into metallic silver grains within voids along the bonded interface. The specimens were embedded in epoxy resin and polished with SiC paper and diamond paste, ultrasonically cleaned, air dried, mounted on stubs, and coated with carbon-gold for the analysis with field-emission scanning electron microscope (FESEM) operated in the backscattered electron mode (JSM 6060, JEOL, Tokyo, Japan). Randomly selected four areas of each sample's hybrid layer were photographed. From the photographs, the total length of the dentin-adhesive interface and the length of interface with silver particles (demonstrating nanoleakage) were measured with ImageJ program (ImageJ 1.42q: NIH, http://rsbweb.nih.gov/ij/index.html). The percentage of hybrid layer length with nanoleakage was calculated.

### Results

The effect of 0.003% DMSO treatment on immediate and long-term nanoleagake, as detected with silver impregnation method is shown in FIG. 10. There is no difference in immediate nanoleakage between DMSO and controls, or between immediate and 6-month results in DMSO group. Six month storage in artificial saliva increased the leakage in the control group (*: statistically significant difference to both immediate control and 6-month DMSO: p<0.05, Mann-Whitney U-test). The results are shown is FIG. 10E.

### Example 5

### DMSO Cytotoxicity on Pulpal Cells

Since the data indicated good penetration abilities of DMSO into dentinal tubules, its cytotoxicity was evaluated using cultured pulpal cells.

### Materials and Methods

Immortalized odontoblast-like MDPC-23 cell line, frequently used for the evaluation of cytotoxicity of dental materials, was used to analyse the effect of different DMSO concentrations on pulpal cell viability. The cell culturing and methodology followed that described by Aranha et al. (2011). Briefly, DMEM culture medium with different concentrations of DMSO (0.05, 0.1, 0.3, 0.5 and 1.0 mM) were placed in contact with MDPC-23 cells (5×104 cells/cm²) for 24 h. Pure DMEM was used in the control group. Twelve replicates were prepared in each group, except for flow cytometry analysis (n=8). Viable cell number (Trypan Blue), cell metabolism (production of succinic dehydrogenase enzyme-SDH, MTT assay) and cell death by necrosis (flow cytometry) were evaluated as described before (Ribeiro et al. 2010, Aranha et al. 2011).

### Results

There were no differences between the groups either in the number of cells or the cell death by necrosis (p>0.05: Table 4). Slight increase was seen with in the production of SDH enzyme by DMSO when compared to the controls, indicating improved cell metabolism (Table 4). However, the increase was not concentration-dependent and may represent normal metabolic variations.

TABLE 4
Effect of different DMSO concentrations on cellular events with
MDPC-23 cells. Viable cell number (Trypan Blue method), cell
metabolism (as measured by production of succinic dehydrogenase
enzyme [SDH] and analysed with MTT assay), and percentage
of cell death by necrosis (flow cytometry).

| DMSO | | | |
|---|---|---|---|
| (mM) | Cell number* | Cell metabolism** | % Cell Death** |
| 0 | 3.9 (1.1) a | 2.315 (2.222-2.351) b | 1.2 (0.7-1.3) a |
| 0.05 | 3.7 (0.9) a | 2.343 (2.287-2.387) ab | 1.2 (0.8-1.6) a |
| 0.1 | 4.3 (1.1) a | 2.468 (2.359-2.557) a | 1.3 (0.9-1.6) a |
| 0.3 | 4.1 (1.4) a | 2.484 (2.399-2.520) a | 1.2 (0.6-1.6) a |
| 0.5 | 4.1 (0.9) a | 2.478 (2.294-2.542) ab | 1.3 (0.8-1.5) a |
| 1.0 | 4.1 (1.3) a | 2.410 (2.119-2.501) ab | 1.3 (0.8-1.6) a |

The values in each column with the same superscript letter did not show statistically significant differences.
**Cell metabolism (MTT assay) and percentage of cell death (flow cytometry): Kruskal-Wallis and Mann-Whitney's test;
*number of viable cells: one-way ANOVA with Tukey's test.

The results clearly show that DMSO is not cytotoxic or cytophatic to odontoblast-like cells even in direct contact. The finding indicates low or non-existing toxicity of DMSO on dental pulp when used with the dental adhesives to improve dentin bonding or with dentin desensitizer, especially since in these dental procedures a dentinal barrier is always present between the used material and pulp tissue. In addition, when used as a carrier of medicaments to regulate inflammatory reaction, reduce pain or to limit the progression of necrotic damage, DMSO will not pose toxic effects to pulp tissue.

### Example 6

### Sealing Capacity of Endodontic Sealers

Since the previous data indicated better wettability achieved with DMSO, fluid filtration method was used to evaluate the leakage in obturated root canals as demonstrated by the fluid flow.

### Materials and Methods

Sixty separate human roots (10/group) from third molars were used for the experiment. The root canals were prepared with rotary endodontic files using NaOCl irrigation. At the end of the preparation, the final irrigation was performed with NaOCl and EDTA in a normal clinical fashion. Then the root canals were irrigated once with either sterile saline (control) or 5% DMSO in sterile saline, dried using paper points, and obturated using gutta percha and one of three sealers using lateral condensation technique: in case of RealSeal the respective gutta percha cones were used as described by the manufacturer. The sealers were allowed to set fully for 24 hours in moist conditions. Then the roots were connected to the Flodec device as described above (Example 2), and subjected to leakage analysis using 10 psi pressure. The recording time was 30 minutes. In case of the leakage exceeding the recording capacity of the device, the highest score measured for the group was given for the sample. All sealers were unaltered commercially available products: TopSeal (from Dentsply Maillefer, Ballaiques, Switzerland), RealSeal (SybronEndo Corp., Orange, Calif., USA) and EndoRez (Ultradent Products, Inc., South Jordan, Utah, USA).

### Results

With all three sealers, pretreatment of DMSO improved the sealing capacity, as demonstrated with lower fluid flow relative to the respective control (FIG. 11). The reduction in the fluid flow was statistically significant with TopSeal (p<0.05, Mann-Whitney U-test) and EndoRez (p<0.01).

The findings clearly demonstrate that DMSO improves the sealing capacity of commercially available and clinically used endodontic sealers, thus improving the impermeability of endodontic obturation. This offers better protection against invading microbes, reducing the possibility of endodontic inflammation in root canal-treated teeth.

### Example 7

### MMP Activity Analyses

### Materials and Methods: Zymography

Gelatin zymography was performed in 10% SDS-PAGE containing 1 mg/ml labelled gelatin in a usual fashion (Invitrogen, Carlsbad, Calif., USA) (O'Grady et al. 1984). After the electrophoresis, SDS was removed by 2.5% Triton X-100 to renature the gelatinases. Gels were then incubated overnight at 37° C. in 50 mM Tris-HCl buffer (5 mM CaCl2, 1 μM ZnCl₂, 0.02% NaN₃, pH 7.5) zymography incubation buffer with 0%, 0.1%, 2.5%, 5%, 10% or 20% of DMSO to observe the effect of DMSO concentration on the MMP activity. The degradation of gelatin was visualized under UV light. Gels were also stained with 0.5% Coomassie Blue R-250. The intensities of the bands were quantified by ScionImage software (Scion Corporation, Frederick, Mass., USA). The analysis was done as triplicates, and relative amounts against 0% DMSO were calculated.

### Materials and Methods: EnzCheck Analysis

EnzChek Gelatinase/Collagenase Assay Kit (Molecular Probes, Eugene, Oreg., USA) was used to detect the effect of DMSO on the gelatinolytic activities of MMP-2 and -9 according to manufacturer's protocol. Briefly, 3 ng of MMP-2 (purified from cell culture media), 3 ng MMP-9 (purified from cell culture media), and 5 ng of recombinant MMP-9 (Chemicon) were diluted into the reaction buffer (50 mM Tris-HCl, 150 mM NaCl, 5 mM CaCl₂, 0.2 mM NaN₃, pH 7.6) and incubated with quenched fluorescein-conjugated gelatin (50 μg/ml) and 0%, 0.01%, 0.1%, 0.5%, 1%, 2%, 3%, 4% or 5% of DMSO in a black-walled 96 well microplate at RT in dark. Fluorescent intensity indicating the gelatinolysis was measured at multiple time points with Fluoromax2/Micromax fluorometer (ISA Jobin Yvon-Spex, Edison, N.J., USA). Analyses were performed as quadruplicates. The relative intensities were calculated against 0% of DMSO.

### Materials and Methods: Hydroxyproline Release

Type I collagen contains about 10 mass % hydroxyproline (HYP), an amino acid almost non-existent in other proteins. Detection of HYP release form demineralized dentin beams was used to evaluate whether DMSO would be effective in inhibiting MMPs in dentin collagen matrix, using a method described in Carrilho et al. 2009. Seventy-six dentin beams (1 mm×1 mm×9 mm) were divided into four groups (n=19 per group) and demineralized. Each beam was individually incubated in a polypropylene tubes with 1 ml of medium containing 50 mmol/L HEPES, 5 mmol/L CaCl2 2H2O, 0.001 mmol/L ZnCl2, 150 mmol/L NaCl, and 3 mmol/L NaN3, pH 7.2, that ensures availability of Zn- and Ca-ions for MMP function. The control samples were incubated in a medium without DMSO: in two groups the beams were incubated with DMSO in medium with 0.001% and 0.01% concentrations. In one group, demineralized dentin beams were immersed in 5% DMSO for 15 minutes and then incubated in a medium without DMSO for the rest of the experiment. Tubes were incubated in a water-shaker bath at 37° C. for a week. Then the medium was completely removed and quantitated for HYP content according to the colorimetric absorbance method described by Jamall et al (1981). Fresh 1 mL of medium was replaced in each tube for additional three weeks of incubation, when the media was removed and analyzed for HYP content. HYP standards were prepared from a 1 mg/mL HYP stock solution in 50% isopropanol. The resulting amount of HYP (mg/mL) was used to estimate the percentage of solubilized (degraded) collagen assuming that 90% of the dry mass of demineralized dentin beams consisted of type I collagen and that 10 mass % of collagen is HYP (Carrilho et al. 2009).

### Results

Since previous studied have indicated that DMSO may inhibit MMP activity (Steffensen et al. 1995), and that MMP inhibition improves long term bond strength (Carrilho et al. 2007a,b, Pashley et al. 2011), the effect of DMSO on MMP activity was examined with gelatin zymography and commercial gelatinase activity kit (EnzChek).

FIG. 12A shows DMSO effect on gelatinases MMP-2 and -9, as measured with gelatin zymography. The disappearance of the dark bands with increasing DMSO concentration clearly demonstrates the inhibitory activity of DMSO on commercially available, highly purified recombinant human gelatinases MMP-2 and MMP-9.

FIG. 12B shows the relative densitometric values of DMSO inhibition of gelatinases in zymographic analysis. The data represents mean±standard deviation (SD) of three individual experiments. The groups with overlapping vertical bars did not show statistically significant differences between each other (p<0.05: ANOVA with Tukey's method).

DMSO effect on gelatinase MMP-2 activity, as measured with commercial enzyme activity kit EnzChek is shown in FIG. 13. 3% DMSO is capable for statistically significant inhibition of the APMA-activated MMP-2. Both in non-activated (no APMA) and activated (APMA) MMP-2 4 to 5% DMSO resulted with theoretical maximum inhibition, as inclusion of control inhibitor provided with the EnzChek kit (phenatroline, PA) did not result with stronger inhibition that 4 or 5% DMSO alone.

DMSO effect on dentin collagenolytic enzymes, as measured with hydroxyproline release from degrading collagen, is shown in FIG. 14. Both concentrations of DMSO (0.001% and 0.01%), as well as 15 minute immersion in 5% DMSO prior the 4-week incubation, significantly decreased HYP release into the medium, demonstrating the inhibition of dentin intrinsic collagenolytic activity by DMSO.

The findings clearly demonstrate that DMSO is capable of inhibiting the MMPs, and the effect is sufficient to eliminate dentin collagen degradation already in low concentrations or after brief exposure to DMSO.

### REFERENCES

Aranha A M, Giro E M, Hebling J, Lessa F C, Costa C A. Effects of light-curing time on the cytotoxicity of a restorative composite resin on odontoblast-like cells. J Appl Oral Sci 2010; 18:461-466.
Breschi L, Mazzoni A, Ruggeri A, Cadenaro M, Di Lenarda R, De Stefano Dorigo E. Dental adhesion review: aging and stability of the bonded interface. Dent Mater 2008; 24:90-101.
Carrilho M R O, Carvalho R M, de Goes M F, di Hipolito V, Geraldeli S, Tay F R, Pashley D H, Tjaderhane L. Chlorhexidine preserves dentin bond in vitro. J Dent Res 2007a; 86:90-94.
Carrilho M R, Geraldeli S, Tay F, de Goes M F, Carvalho R M, Tjaderhane L, Reis A F, Hebling J, Mazzoni A, Breschi L, Pashley D H. In vivo preservation of the hybrid layer by chlorhexidine. J Dent Res 2007b; 86:529-533.
Carrilho M R, Tay F R, Donnelly A M, Agee K A, Tjaderhane L, Mazzoni A, Breschi L, Foulger S, Pashley D H. Host-derived loss of dentin matrix stiffness associated with solubilization of collagen. J Biomed Mater Res B Appl Biomater 2009; 90:373-80.
Carrilho M R, Tay F R, Pashley D H, Tjaderhane L, Carvalho R M. Mechanical stability of resin-dentin bond components. Dent Mater 2005; 21:232-241.
Cooper P R, Takahashi Y, Graham L W, Simon S, Imazato S, Smith A J. Inflammation-regeneration interplay in the dentine-pulp complex. J Dent 2010; 38:687-697.
Klein-Júnior C A, Zander-Grande C, Amaral R, Stanislawczuk R, Garcia E J, Baumhardt-Neto R, Meier M M, Loguercio A D, Reis A. Evaporating solvents with a warm air-stream: effects on adhesive layer properties and resin-dentin bond strengths. J Dent 2008; 36:618-625.
De La Macorra J C, Escribano N I. Comparison of two methods to measure permeability of dentin. J Biomed Mater Res 2002; 63:531-534.
Jamall I S, Finelli V N, Que Hee S S. A simple method to determine nanogram levels of 4-hydroxyproline in biological tissues. Anal Biochem 1981; 112:70-75.
O'Grady R L, Nethery A, Hunter N. A fluorescent screening assay for collagenase using collagen labelled with 2-methoxy-2,4-diphenyl-3(2H)-furanone. Anal Biochem 1984; 140:490-494.
Pashley D H, Tay F R, Breschi L, Tjaderhane L, Carvalho R M, Carrilho M, Tezvergil-Mutluay A. State of the art etch-and-rinse adhesives. Dent Mater 2011; 27:1-16.
Ribeiro A P, Pavarina A C, Trindade F Z, Inada N M, Bagnato V S, de Souza Costa C A. Photodynamic therapy associating Photogem and blue LED on L929 and MDPC-23 cell culture. Cell Biol Int 2010; 34:343-351.
Steffensen B, Wallon U M, Overall C M. Extracellular matrix binding properties of recombinant fibronectin type II-like modules of human 72-kDa gelatinase/type IV collagenase. High affinity binding to native type I collagen but not native type IV collagen. J Biol Chem 1995; 270:11555-11566.
Tay F R, Gwinnett J A, Wei S H. Relation between water content in acetone/alcohol-based primer and interfacial ultrastructure. J Dent 1998; 26:147-156.
Tersariol I L, Geraldeli S, Minciotti C L, Nascimento F D, Paakkonen V, Martins M T, Carrilho M R, Pashley D H, Tay F R, Salo T, Tjaderhane L. Cysteine cathepsins in human dentin-pulp complex. J Endod 2010; 36:475-481.
Van Landuyt K L, Snauwaert J, De Munck J, Peumans M, Yoshida Y, Poitevin A, Coutinho E, Suzuki K, Lambrechts P, Van Meerbeek B. Systematic review of the chemical composition of contemporary dental adhesives. Biomaterials 2007; 28:3757-3785.
Zehnder M. Root canal irrigants. J Endod 2006; 32:389-398.

1. A dental composition for restoration or decoration of teeth comprising
(a) at least one monomer; and optionally
(b) a solvent and
(c) one or more constituent(s) selected from the group consisting of initiators, inhibitors, desensitizing agents, fillers, silane coupling factors, cross-linking agents, dyes and acids,
wherein the composition further comprises 0.0001-1.9% DMSO.

2. The composition of claim 1 wherein the monomer is selected from a group consisting of 4-acryloyloxyethyl trimellitate anhydride (4-AETA), 4-acryloylethyl trimellitic acid (4-AET), 2-acrylamido-2-methyl-1-propanesulfonic acid (AMPS), bis[2-(methacryloyloxy)-ethyl]phosphate (Bis-MEP), ethoxylated bisphenol A glycol dimethacrylate (bis-EMA), bisphenol A diglycidyl methacrylate (bis-GMA), biphenyl dimethacrylate or 4,40-dimethacryloyloxyethyloxycarbonylbiphenyl-3,30-dicarboxylic acid (BPDM), di-2-hydroxyethyl methacryl hydrogenphosphate (di-HEMA phosphate), dimethylaminoethyl methacrylate (DMAEMA), ethyl 2-[4-(dihydroxyphosphoryl)-2-oxabutyl]acrylate (EAEPA), ethyleneglycol dimethacrylate (EGDMA), glycerol dimethacrylate (GDMA), glycerol phosphate dimethacrylate (GPDM), 1,6-hexanediol dimethacrylate (HDDMA), 2-hydroxyethyl methacrylate (HEMA), 2-hydroxyethyl methacryl dihydrogenphosphate (HEMA-phosphate), hexafluoroglutaric anhydride-glycerodimethacrylate adduct (HFGA-GMA), 2-hydroxypropyl methacrylate (HPMA), methacrylic acid (MA), 2,4,6 trimethyl-phenyl 2-[4-(dihydroxyphosphoryl)-2-oxabutyl]acrylate (MAEPA), 11-methacryloyloxy-1,10-undecanedicarboxylic acid (MAC-10), 10-methacryloyloxydecyl dihydrogen-phosphate (10-MDP), methacryloyloxydodecylpyridinium bromide (MDPB), 4-methacryl-oyloxyethyl trimellitate anhydride (4-META), 4-methacryloyloxyethyl trimellitic acid (4-MET), methyl methacrylate (MMA), mono-2-methacryloyloxyethyl phthalate (MMEP) which is sometimes also called phtalic acid monomethacrylate (PAMA), N-methacryloyl-5-amino-salicylic acid (5-NMSA or MASA), N-phenylglycine glycidyl methacrylate (NPG-GMA) and N-tolylglycine glycidyl methacrylate and N-(2-hydroxy-3-((2-methyl-1-oxo-2-propenyl)oxy)propyl)-N-tolyl glycine (NTG-GMA).

3. A method of improving long term adhesion of compositions or components to dental tissues comprising including DMSO into one or more composition.

4. The method of claim 3 wherein DMSO is included to dental composition such as an etchant (demineralizing agent), a wetting agent, a primer, an adhesive or any combination thereof.

5. The method of claim 4 wherein DMSO concentration is 0.0001 to 50%, preferably 0.0005 to 30%, more preferably 0.001 to 10% and most preferably 0.003 to 5%.

6. The method of claim 3 comprising the steps of
(a) etching,
(b) priming,
(c) applying adhesive and
(d) introducing a component to be bonded to the surface of tooth;
wherein the steps may be either separate or combined, and wherein DMSO is included in to a dental composition in at least one of the steps.

7. The method of claim 3, wherein DMSO is included into one-step filling composition such as self-etching cement.

8. The method of any of claim 3, wherein DMSO is included into priming or adhesion step or into both printing and adhesion steps.

9. The method of claim 3, wherein a filling material is introduced.

10. A dental composition for use in pulp medication comprising
one or more constituents of the group consisting of MMP inhibitors, fluoride, anti-microbial agents, anti-inflammatory agents, growth factors and pain medicaments; and optionally a solvent wherein the composition further comprises 0.0001-80 w % DMSO.

11. A dental composition for root canal disinfection and/or obturation comprising
(a) at least one disinfectant or demineralizing agent; and optionally
(b) additional antimicrobial or demineralizing agents;
(c) detergents,
wherein the composition further comprises 0.0001-80% DMSO.

12. A dental composition for root canal obturation comprising
(a) an irrigant or primer and a sealer; or
(b) a sealer,
wherein the composition further comprises 0.0001-1.9% DMSO.

13. A dental composition for desensitizing comprising
two or more of the group consisting of ions or salts, protein precipitant(s) and dentin sealer(s); and
optionally one or more of the group consisting of further solvents, detergents and homeopathic medications
wherein the composition further comprises 0.0001-80% DMSO.

14. (canceled)

15. A method of using DMSO in preparing dental compositions having improved long term adhesion to dentin, enamel or cement.

16. The method of claim 15 wherein the composition is a primer or adhesive or combination thereof.

17. A method of improving penetration of compositions to dentin, enamel, dental pulp or cement comprising including DMSO into one or more composition.

18. The method of claim 17 wherein DMSO is included to a solution for pulpal treatment, a desensitizing agent or an agent for treating root canal.

19. The method of claim 17, wherein DMSO concentration is 0.0001 to 80% in solutions for pulpal treatment, desensitizing agent or root canal treatment.

20. A method of treating teeth comprising using DMSO to improve long term adhesion of components and optionally to inhibit MMP's.
